(19) United States
(12) Patent Application Publication
(10) Pub. No.: US 2006/0265771 A1
(43) Pub. Date: Nov. 23, 2006

(54) Monitoring microrna expression and function

(76) Inventors: David L. Lewis, Madison, WI (US); Thomas W. Reppen, Madison, WI (US); Paula L. Roesch, Oregon, WI (US)

Correspondence Address: MIRUS CORPORATION, 505 SOUTH ROSA RD, MADISON, WI, 53719, US

(21) Appl. No.: 11/412,154

(22) Filed: Apr. 26, 2006

Related U.S. Application Data
(60) Provisional application No. 60/681,886, filed on May 17, 2005.
(60) Provisional application No. 60/711,080, filed on Aug. 24, 2005.

(51) Int. Cl. A01K 67/027; C40B 30/06; C40B 40/08

(52) U.S. Cl. 800/18; 435/6

(57) ABSTRACT

In vivo endogenous microRNA (miRNA) activity can be observed over time using miRNA sensor plasmids capable of long term expression. Using reporter genes whose expression can be monitored without sacrificing the animal enables the investigator to follow changes in miRNA expression though developmental stages or in response to environmental factors or treatment regimens.

### CROSS-REFERENCE TO RELATED APPLICATIONS

This application claims the benefit of U.S. Provisional Application No. 60/674,504, filed Apr. 28, 2005, U.S. Provisional Application No. 60/681,886, filed May 17, 2005, and U.S. Provisional Application No. 60/711,080, filed Aug. 24, 2005.

### BACKGROUND OF THE INVENTION

Recently, much interest has focused on a recently discovered population non-coding small RNA molecules, termed small interfering RNA (siRNA) and micro RNA (miRNA), and their effect on intracellular processes, particularly gene expression. Micro RNAs (miRNAs) and small interfering RNAs (siRNAs) are small RNAs about 15-50 nucleotides in length which play a role in regulating gene expression in eukaryotic organisms.

Most genes function by expressing a protein via an intermediate, termed messenger RNA (mRNA) or sense RNA. RNA interference (RNAi) describes a phenomenon whereby the presence of double-stranded RNA (dsRNA) of sequence that is identical or highly similar to sequence in a target gene mRNA results in inhibition of expression of the target gene. It has been found that RNAi in mammalian cells can be mediated by short interfering RNAs (siRNAs) of typically about 18-25 nucleotides (base pairs) in length. Functional siRNAs can be synthesized chemically or they can be formed endogenously through processing of long double strand RNA or transcription of siRNA encoding transgenes.

More recently, a class of endogenous small RNA molecules has been discovered, termed microRNAs. MicroRNAs (miRNAs) are a family of short, non-coding RNAs that are thought to regulate post-transcriptional gene expression through sequence-specific base pairing with target mRNAs in a manner similar to RNAi. They are expressed in a wide variety of organisms ranging from plants to worms and humans. Thus far, more than 800 miRNAs have been identified in humans, with many being conserved in other mammalian species.

Some miRNAs are transcribed as long primary transcripts (pri-miRNA). They can be embedded in independent noncoding RNAs or in introns of protein-coding genes. After the pri-miRNAs are processed into the small miRNAs, the mature miRNAs get assembled into the effector complexes called miRNPs (miRNA-containing ribonucleo-protein particles) that share significant similarity to RISC, the complex which mediates siRNA action.

Once the miRNP is assembled, the miRNA guides the complex to its target by base-pairing with the target mRNA. Most animal miRNAs bind to multiple, partially complementary binding sites in the 3′-UTRs of the target genes. However, binding site sequences inserted into either coding or 5′-UTR sequences are also functional. The fate of the target mRNA may be decided by the extent of base-pairing to the miRNA. Evidence suggests that miRNA will direct destruction of the target mRNA, gene silencing, if it has perfect or near-perfect complementarity to the target. On the other hand, the presence of multiple, partially complementary sites in the target mRNA may result in translation repression without strongly affecting mRNA levels though inhibit of protein accumulation on the transcript.

MiRNAs appear to be a major feature of the gene regulatory networks of animals. Roles for miRNAs have been suggested in development, embryogenesis and patterning, differentiation and organogenesis, growth control and programmed cell death, and even human disease, including cancer and inhibition of viral replication. The specific targets for miRNAs are largely unknown, but thousand of genes may be so regulated. It is also possible that a given mRNA may be targeted by multiple miRNAs or that a given miRNA may regulate multiple mRNAs.

In animals, miRNA has been proposed to primarily fine-tune gene expression and to dramatically regulate the expression of a much smaller number of transcripts. Several miRNAs are expressed in a tissue-specific and developmental stage-specific manner. In fact, it was shown that the miRNA profiles are changed in a large number of cancers and that the forced overexpression of miRNAs can lead to the development of tumors.

Currently, the function of individual microRNAs has been analyzed using time-intensive procedures, including cloning, Northern hybridization, and microarray analysis. Bioinformatic prediction of animal miRNA targets is complicated by the only modest complementarity animal miRNAs have to their targets. MiRNA profiling has also been used to identify miRNAs with potentially important developmental roles. The rationale is that if a miRNA is highly expressed in a tissue or cell type or at a specific developmental stage, it may reasonably expected to play a regulatory role in specifying tissue or cell identity, or in regulating developmental timing. While these methods can demonstrate the presence of a miRNA in a cell, they do not yield information regarding whether or not the miRNA is functional.

Smirnova et al. and Mansfield et al. have used miRNA-sensitive sensor transgenes to detect the presence and function of miRNA in cells. These miRNA sensor transgenes contained miRNA binding sites on reporter gene mRNAs, rendering expression of the reporter gene sensitive to the presence of the miRNA. Smirnova et al. used miRNA sensor plasmids to analyze the expression of miR-125 and miR-128 in primary cortical neurons and astrocytes in vitro in order to confirm neuron-specific expression. Mansfield et al. used miRNA sensor constructs to examine expression of miRNA in transiently transgenic mouse embryos. While offering a simpler method of assessing miRNA function in a given cell type, these sensor transgenes sacrifice of the animal to obtain miRNA function information. The availability of a method to quickly and easily monitor miRNA expression in adult animals over time would greatly facilitate studies focused on the in vivo role of miRNAs.

### SUMMARY OF THE INVENTION

We describe the creation and intended use of a miRNA sensor plasmid and related library that provides for spatial and temporal detection of miRNA expression and function in the liver of adult mice. The miRNA sensor plasmid comprises: a sequence complementary to a known or suspected miRNA, a miRNA binding or target sequence, located in the 3′ UTR of an expression cassette capable of long term hepatocyte expression of a secreted detectable reporter protein. The expression cassette is delivered, optionally along with a control reporter gene, to a cell in vivo. If the miRNA is expressed and active in the cell, translation of the transcribed reporter gene into the protein product is inhibited.

The reported protein comprises a protein that can be readily detected using methods known in the art without sacrificing the animal. A preferred reporter protein is a secreted protein detectable in the serum since blood can be drawn from an animal multiple times over the course of days, weeks, months or even years without sacrificing or harming the animal. A preferred protein is also minimally immunogenic. An example of a preferred reporter protein is secreted alkaline phosphatase (SEAP). For analysis of miRNA in a mouse, it is preferred to use mouse SEAP. Another preferred reporter protein is a soluble version of CD4, especially mouse CD4.

In one embodiment, the miRNA sensor plasmid contains a reporter gene expression cassette that encodes a secreted reporter protein and contains transcription elements capable of long term expression of the reporter gene. An exemplary expression cassette is described in U.S. application Ser. No. 10/229,786, which is incorporated herein by reference. A preferred expression cassette comprises an α-fetoprotein enhancer and an albumin promoter. A preferred expression cassette further comprises a 5′ intron. Exemplary 5′ introns include, but are not limited to, the chimeric intron (from the pCI Mammalian Expression Vector, Promega, Madison, Wis.) and the human factor IX intron. A preferred expression cassette further comprises a 3′ UTR intron. An exemplary 3′ UTR intron is a truncated intron 14 from the human albumin 3′UTR. A preferred expression cassette further comprises one or more perfectly matched miRNA binding sites. The miRNA binding sites may also include binding sites that are not perfectly matched. The miRNA binding sites are preferably located in the 3′ UTR of the reporter gene expression cassette, but may also be located in other regions of the expression mRNA. To further reduce immunogenicity of the report plasmid, the plasmid can be optimized to reduce or eliminate CpG dinucleotides. The miRNA sensor plasmid may further comprise a second expression cassette that encodes a control reporter protein. Alternatively, a control reporter protein may be expressed from a gene on a separate plasmid and delivered together with the miRNA sensor plasmid.

Long term expression of the reporter gene allows the investigator to monitor changes in miRNA expression or activity over time. Having a reporter protein that is secreted and detectable in the blood eliminates the need to sacrifice the animal or tissue, therefore allowing the investigator to monitor miRNA expression of function over time in the same animal. These features permit one to determine if miRNAs are differentially active or expressed under different conditions, such as disease state, infection, fasting, response to changing environmental or developmental conditions, etc.

The miRNA sensor plasmid can be delivered to hepatocytes in an animal using gene delivery methods practiced in the art. Known gene delivery methods include: hydrodynamic intravascular delivery, including hydrodynamic tail vein injection, direct parenchymal injection, biolistic transfection, electroporation, lipid transfection (lipofection), polycation mediated transfection (polyfection), and lipid-polycation complex mediated transfection (lipopolyfection). A preferred delivery method is hydrodynamic tail vein (HTV) injection. HTV injection provides a rapid, easy, reliable, nonsurgical method of polynucleotide delivery to the liver (U.S. Patent 6,627,616, incorporated herein by reference). Another preferred delivery method is hydrodynamic limb vein (HLV) injection (U.S. patent application, incorporated herein by reference).

### BRIEF DESCRIPTION OF THE FIGURES

FIG. 1. Diagram of a short term miRNA sensor plasmid.

FIG. 2. Diagram of a long term miRNA sensor plasmid encoding a secreted alkaline phosphatase reporter protein.

FIG. 3. Graph illustrating comparison of expression of secreted alkaline phosphatase in C57B1/6 mouse liver from a different expression cassettes delivered by hydrodynamic tail vein injection. Open circles represent SEAP expression under the control of the CMV immediate early viral enhancer is shown (CMV). Black squares represent SEAP expression from the described long term expression cassette. n=8, error bars represent SEM.

FIG. 4. Bar graph illustrating detection of miRNA activity, as measure by inhibition of reporter gene expression from miRNA sensor plasmids, in mouse liver and muscle cells. Data are plotted as the ratio of target Renilla luciferase activity (Rr-Luc) to control firefly luciferase activity (Pp-Luc+) normalized to Renilla luciferase activity from a no miRNA binding site control plasmid (none). n=3, error bars represent SD.

FIG. 5. Bar graph illustrating comparison of inhibition of reporter gene expression in mouse liver from miRNA sensor plasmids containing wild type (WT) vs. mutant miRNA binding site sequences. Mutations at base positions 3, 7 and 10 in miRNA binding sites disrupt inhibition by endogenous miRNAs. n=3, error bars represent SD.

FIG. 6. Bar graph illustrating effectiveness of detecting miRNA activity in HeLa cells in culture using miRNA sensor plasmids. The graph further shows blocking of miRNA activity using antisense oligonucleotides. MiRNA sensor plasmids are described in Table 2. Antisense oligonucleotides are described in Table 3.

FIG. 7A-7D. Bar graphs illustrating analysis of inhibition of miRNA activity by antisense oligonucleotides. MiRNA sensor plasmids containing the indicated miRNA binding sites were delivered to mouse liver cells together with control or an anti-miRNA oligonucleotides specific for the indicated miRNA. A. Effect of anti-miR-18 and anti-miR-192 oligonucleotides on expression of pMIR394 and pMIR395 miRNA sensor plasmids. B. Effect of anti-miR-1, anti-miR-122 and anti-miR-143 oligonucleotides on expression of pMIR399, pMIR399 and pMIR400 miRNA sensor plasmids. C. Effect of antisense miRNA oligonucleotides on expression of reporter genes containing the cognate miRNA binding sites. D. Inset showing data for the miR-122a sensor plasmid from C.

FIG. 8. Bar graph illustrating comparison of different oligonucleotides chemistries in inhibiting miR-122a activity in the liver in vivo. Comparison of 2′-OMe, morpholino, locked nucleic acid (LNA) oligonucleotides is shown. n=3, error bars represent SD.

### DETAILED DESCRIPTION

Determining the expression patterns of miRNAs within specific cell and tissues types is important in understanding how miRNAs function in cell biology. Techniques such as Northern blot analysis, strategic cloning, microarray profiling, and quantitative PCR have allowed investigators to determine which miRNAs are present in a cell or tissue of interest. However, these methods require destroying the cells or tissues of interest and isolating cellular RNA, thereby prohibiting the investigator from monitoring changes in miRNA expression in the same animal over time and under different conditions. These methods also address presence, but not necessarily activity, of the miRNAs. The described miRNA sensor system provides a facile, user-friendly system for detection of miRNA activity that does not require the animal to be sacrificed.

Detection of miRNA activity is based on analysis of expression of a reporter gene that contains a miRNA binding site, preferable within the 3′ UTR of the reporter gene. If the cognate miRNA is expressed and functional in a cell, the miRNA will inhibit expression of the reporter gene. Inhibition of gene expression refers to an detectable decrease in the level of protein and/or mRNA product from a reporter/target gene. The level of inhibition of reporter gene activity can indicate the level of miRNA that is active in the cell. The reporter gene is expressed from a miRNA sensor plasmid which is delivered to cells in a desired tissue in an animal. The described miRNA sensor plasmids are capable of long term expression of a reporter gene which encodes a secreted protein. By using a reporter protein that is secreted into the circulation, it is possible to monitor miRNA at multiple time points in a single animal. By using a sensor plasmid capable of long term expression of the reporter gene, the described miRNA sensor system allows an investigator to monitor changes in miRNA activity over time in the same animal under a variety of treatment, environmental or developmental conditions.

The miRNA sensor plasmid comprises an expression cassette which a) encodes a reporter protein, b) enables long term expression of the reporter gene and c) contains a miRNA binding site.

In one embodiment, the miRNA sensor plasmid that contains elements that allow for long-term expression of a transgene in liver as described in U.S. application Ser. No. 10/229,786. The liver may be of particular interest to biological researchers because there is evidence that liver miRNAs are involved in metabolic processes and their expression may be modulated with changes in metabolic status. In this embodiment, the sensor plasmid contains a liver-specific, a long-term enhancer/promoter combination. A preferred long-term enhancer/promoter combination is the albumin promoter together with the alpha-fetoprotein enhancer element. Other promoter/enhancer elements may be more appropriate for other long term expression in cell types in other tissues. The described liver-specific long term expression vector further comprises a 5′ intron and a 3′ intron. The 3′ UTR intron is located less than about <50 nucleotides downstream of the expression cassette translation stop codon. The 3′ intron is positioned to avoid non-sense mediated decay of the reporter gene mRNA. Using the described long term expression cassette, expression in the liver was been observed to be high for at least 14 months.

The miRNA sensor plasmid contains a reporter gene which encodes a reporter protein. A reporter protein is a protein that can be quantitatively detected using methods known in the art. Typically, reporter proteins include enzymes, fluorescent proteins, and proteins or peptides that can be readily detected with antibodies. Enzymes are those proteins whose enzymatic activity can be measured. Reporter proteins commonly used in the art include both intracellular and secreted proteins. Examples include, but are not limited to: luciferase, β-galactosidase, chloramphenicol acetyl transferase, green fluorescent protein (and variants thereof), growth hormone, factor IX, secreted alkaline phosphatase, alpha 1-antitrypsin, and soluble CD4. For the present invention, secreted reporter genes are preferred. More specifically, secreted proteins which can be detected in blood samples are preferred.

Immune response to the reporter gene can be a limiting factor in obtaining long term expression. Therefore, the use of minimally or non-immunogenic reporter proteins are preferred. Using a reporter protein that is native to the investigated species reduces the likelihood of an immune reaction against the reporter protein. For example, for monitoring miRNA activity in mouse, murine secreted alkaline phosphatase (mSEAP) is a preferred reporter protein. Whether a given reporter protein elicits an immune response in a given strain of a given species can be determined using methods known in the art; detecting antibodies or immune cells specific to the protein. The use of reporter genes that are non-immunogenic and are secreted into the bloodstream enable an investigator to monitor miRNA expression by taking blood samples and using simple assays for reporter gene expression. SEAP expression can be assayed in multi-well plates using commercially available chemiluminescent reagents. Soluble mouse CD4 (smCD4) can be assayed by Enzyme Linked ImmunoSorbant Assay (ELISA) using commercially available antibodies (Abcam).

A miRNA binding site is a nucleotide sequence which is complementary or partially complementary to at least a portion of a miRNA. The sequence can be a perfect match, meaning that the binding site sequence has perfect complementarity to the miRNA. Alternatively, the sequence can be partially complementary, meaning that one or more mismatches may occur when the miRNA is base paired to the binding site. Partially complementary binding sites preferably contain perfect or near perfect complementarity to the seed region of the miRNA. The seed region of the miRNA consists of the 5′ region of the miRNA from about nucleotide 2 to about nucleotide 8. For naturally occurring miRNAs and target genes, miRNAs with perfect complementarity to an mRNA sequence direct degradation of the mRNA through the RNA interference pathway while miRNAs with imperfect complementarity to the target mRNA direct translational control (inhibition) of the mRNA. The invention is not limited by which pathway is ultimately utilized by the miRNA in inhibiting expression of the reporter gene.

The miRNA binding site is preferably located in the 3′ untranslated region (UTR) of the reporter gene mRNA. In one embodiment, the miRNA binding site(s) are positioned just downstream of a 3′ UTR intron and about 100 nucleotides upstream of a polyadenylation signal. To facilitate cloning of a miRNA binding site into the miRNA sensor expression cassette, one or more restriction endonuclease sites are inserted into the 3′ UTR at the site of insertion of the miRNA binding site. In one embodiment, the miRNA sensor plasmid contains a liver-specific long term expression cassette encoding the murine SEAP gene in which an exact match miRNA binding site is inserted into the 3′ UTR.

A control expression cassette encoding a second control reporter protein may be co-delivered with the miRNA sensor plasmid. The control reporter protein serves as an internal reference to normalize delivery efficiency of the miRNA sensor gene. A preferred control reporter protein comprises the soluble version of mouse CD4 (smCD4). The control expression cassette can be present on the same plasmid as the miRNA sensor gene, or it may be located on an independent plasmid which is co-delivered.

In one embodiment, a miRNA sensor plasmid library is formed. A miRNA sensor library comprises a set of miRNA sensor plasmids with independent and unique miRNA binding sites. A library may contain miRNA sensor plasmids for each of the known or suspected miRNAs in a species, in a specific tissue or cell type, or present at a specific developmental stage. In a preferred embodiment, the miRNA sensor library contains an exact match miRNA biding site for each desired miRNA. The availability of such a library will enable examination of expression of any known miRNA. Lists of known miRNA sequences can be found in databases maintained by research organizations such as the Wellcome Trust Sanger Institute. The current number of known or suspected mouse miRNAs is more that 200 (miRBase release 7.1).

A preferred delivery method is non-viral hydrodynamic intravascular delivery. Hydrodynamic gene delivery is well known in the art and comprises rapidly injecting the polynucleotide in a large volume into an afferent of efferent vessel of a target tissue. Hydrodynamic intravascular delivery has been shown to be efficient for naked polynucleotides, polynucleotides complexed with non-viral delivery agents and for viruses. For delivery to hind limb skeletal muscle, the polynucleotide can be injected into an artery, such as the femoral artery, or a vein, such as the saphenous vein. For delivery to liver, the polynucleotide can be injected into the hepatic artery or vein, portal vein, bile duct, or tail vein. Hydrodynamic tail vein injection for delivery to mouse or rat liver is a preferred method because the delivery does not require a surgical procedure.

Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

The term expression cassette refers to a naturally, recombinantly, or synthetically produced nucleic acid molecule that is capable, of expressing a gene or genetic sequence in a cell. An expression cassette typically includes a promoter (allowing transcription initiation), and a sequence encoding one or more proteins or RNAs. Optionally, the expression cassette may include transcriptional enhancers, non-coding sequences, splicing signals and introns, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences. Optionally, the expression cassette may include a gene or partial gene sequence that is not translated into a protein.

The term gene generally refers to a nucleic acid sequence that comprises coding sequences necessary for the production of a nucleic acid (e.g., siRNA) or a polypeptide (protein) or protein precursor. A polypeptide can be encoded by a full length coding sequence or by any portion of the coding sequence so long as the desired activity or functional properties (e.g., enzymatic activity, ligand binding, signal transduction) of the full-length polypeptide or fragment are retained. In addition to the coding sequence, the term gene may also include, in proper contexts, the sequences located adjacent to the coding region on both the 5′ and 3′ ends which correspond to the full-length mRNA (the transcribed sequence) or all the sequences that make up the coding sequence, transcribed sequence and regulatory sequences. The sequences that are located 5′ of the coding region and which are present on the mRNA are referred to as 5′ untranslated region (5′ UTR). The sequences that are located 3′ or downstream of the coding region and which are present on the mRNA are referred to as 3′ untranslated region (3′ UTR). The term gene encompasses synthetic, recombinant, cDNA and genomic forms of a gene. A genomic form or clone of a gene contains the coding region interrupted with non-coding sequences termed introns, intervening regions or intervening sequences. Introns are segments of a gene which are transcribed into nuclear RNA. Introns may contain regulatory elements such as enhancers. Introns are removed or spliced out from the nuclear or primary transcript; introns therefore are absent in the mature mRNA transcript. Regulatory sequences include, but are not limited to, promoters, enhancers, transcription factor binding sites, polyadenylation signals, internal ribosome entry sites, silencers, insulating sequences, matrix attachment regions. Non-coding sequences may influence the level or rate of transcription and/or translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, nuclear transport, and immunogenicity. Gene expression can be regulated at many stages in the process. Up-regulation or activation refers to regulation that increases the production of gene expression products (i.e., RNA or protein), while down-regulation or repression refers to regulation that decrease production. Molecules (e.g., transcription factors) that are involved in up-regulation or down-regulation are often called activators and repressors, respectively.

Long term expression means that the gene is expressed for greater than 2 weeks, greater than 4 weeks, greater than 8 weeks, greater than 20 weeks, greater than 30 weeks, or greater than 50 weeks with less than a 1 0-fold decrease in expression from day 1. Expression from typical CMV promoter driven gene expression cassettes typically drops by up to 1 000-fold after 7 days. Expression for longer than a few weeks may require not eliciting an immune response to the expressed gene product, which is independent of the promoter/enhancer elements of the expression cassette. An immune response can be avoided or minimized by using immunosuppressive drugs, immune compromised animals, or expressing a gene product that is minimally or non-immunogenic.

The generation of mice containing miRNA sensor plasmids using hydrodynamic delivery has several advantages over other methodologies such as the generation of miRNA sensor knock-in (transgenic) mice. Hydrodynamic injection is a relatively simple procedure that can be performed with minimal training and does not require special skills, equipment, cell lines or reagents. Using hydrodynamic injection, an investigator can generate miRNA sensor mice quickly and for a large number of different miRNAs. Generation of a transgenic mouse takes at least 8 weeks and it is a matter of months before enough mice are available to perform an experiment. The hydrodynamic injection method can also be used on any mouse strain, whereas knock-in technology is practically limited to just a few. Thus, hydrodynamic delivery of long term expression miRNA sensor plasmids can be used in specialized mouse strains or disease models not amenable to transgenic technology. Also, with minor modification of the plasmids, the system can be used in rats or other animals. Also, because expression of the reporter gene from the described expression vectors persists for more than a year, these sensor plasmids can be used to generate transfected mice which can then be distributed to other researchers.

The described miRNA sensor system can be used to study differences in miRNA activity in development, cellular differentiation, and metabolism. Currently, it is known that certain miRNAs are differentially expressed under different conditions or developmental stages. In mice, there is evidence that the pancreatic islet specific miR-375 plays a role in glucose stimulated insulin secretion. Studies performed using murine pancreatic b-cell line MIN6 indicate that increasing the cellular levels of miR-375 by transfection of synthetic miR-375 or infection with adenovirus overexpressing miR-375 decreased insulin secretion in response to glucose stimulation. Conversely, antisense inhibition of endogenous miR-375 increased insulin secretion. Depletion of one of the miR-375 targets, myotropin, by RNAi, was shown to reduce insulin secretion. Together these results suggest that miR-375 affects insulin secretion from pancreatic islet cells at least in part by repressing the expression of myotropin. A role for miRNAs in cholesterol homeostasis has also been described. The miRNA, miR-122, is specifically expressed in the liver at very high levels. Microarray analysis of liver mRNAs differentially expressed upon delivery of antisense miR-122 oligonucleotides identified cholesterol biosynthesis genes as being down-regulated. In agreement with this finding, treatment with antisense miR-122 resulted in about a 40% decrease in plasma cholesterol levels. Furthermore, we have discovered, through miRNA microarray analyses, that expression of some miRNAs in the liver are modulated in response to fasting.

The long term expression miRNA sensor plasmids can be used to study differential expression and activity of these and other miRNAs is response to a variety of developmental and environmental conditions using a simple, blood-based assay. The analysis of expression patterns of miRNAs can also provide clues as to their possible function and can be used to understand the function of miRNA in regulation of gene expression, including developmentally important gene or genes important in metabolism or disease.

The long term expression miRNA sensor plasmids can be used to investigate anti-miRNA molecules. MiRNA sensor plasmid can be used to evaluate the effectiveness of different types of miRNA inhibitors, including antisense miRNA oligonucleotides. The effectiveness of different oligonucleotide chemistries or modifications, in blocking miRNA activity, can be measured. Different oligonucleotide chemistries have been developed to enhance their activity. The miRNA sensor genes provide a rapid, reliable method to assess their effectiveness in vivo.

The use of anti-miRNA molecules targeting the endogenous miRNA of interest can provide a means to confirm results obtained from the miRNA sensor plasmid. If inhibition of the miRNA sensor gene is due to the presence of the cognate miRNA, co-delivery of the anti-miRNA molecule will result in relief of inhibition of reporter gene expression from the miRNA sensor plasmid. Antisense oligonucleotides complementary to endogenous miRNAs have been shown to transiently block miRNA function and therefore can be utilized and anti-miRNA molecules.

It is also possible to use an endogenous miRNA as a means of regulating expression of a transgene. By constructing a plasmid that encodes a gene of interest, instead of a reporter gene, and placing a specific miRNA binding site in the gene of interest, expression of the gene becomes sensitive to the miRNA phenotype of the cell-type to which the plasmid is delivered.

As an example, a plasmid can be constructed that codes for a toxic protein such as tumor necrosis factor-α (TNFα). A specific miRNA binding site can be placed in the 3′ UTR of the TNFα. If the plasmid is delivered to a cell that contains the cognate miRNA, the miRNA will inhibit expression of the TNFαgene in that cell. However, if the same plasmid is delivered to a cell that does not contain the cognate miRNA, TNFα is expressed, resulting in decreased viability of the cell. An example of a miRNA that is specifically present in a given cell type is the miR-122 miRNA, which is normally present in high levels in animal liver cells. Delivery of a plasmid encoding a transgene containing a miR-122 site to a normal liver cell would result in repression of the transgene in that cell. In contrast, delivery of the same plasmid to a cell that does not express miR-122, would result in expression of the transgene, such as TNFα, in that cell. In this way, a cancer cell; or other desired cell, may be selectively targeted for expression of the transgene, by selecting a miRNA binding site that corresponds to a miRNA that is not expressed in the target cell, but is expressed in surrounding cells.

In a similar method, the process can be used to target expression of a transgene in cells that have a high level of a particular miRNA and while neighboring or non-target cells have little or none. For this process, a gene encoding a repressor or inhibitor of the transgene or encoded protein is co-delivered to the cell, preferably by encoding the repressor/inhibitor on the same plasmid as the transgene. By placing a miRNA binding site in the gene sequence of the repressor/inhibitor gene, expression of the repressor/inhibitor is dependent on the presence or absence of the cognate miRNA in the cell. If the plasmid is delivered to a cell of interest and the miRNA is present in the cell, the miRNA binds and causes inhibition of expression of the repressor/inhibitor mRNA. By reducing or eliminating expression of the repressor/inhibitor, expression or activity of the transgene is increased. Expression of the transgene in non-target cells is reduced because of the absence the miRNA, resulting in expression of the repressor/inhibitor and therefore repression or inhibition of the transgene.

As an example illustrating the process, a plasmid can be constructed that contains a TNFα repressor such as heat shock factor 1, in addition to the TNFα gene. A miRNA binding site is placed in the of the HSF-1 gene, wherein the miRNA is known to be expressed in the target cell, but not in non-target cells to which the plasmid may be delivered. If the plasmid is delivered to the desired targeted cells, the miRNA binds, expression of the repressor mRNA is inhibited and TNFα is expressed by the plasmid. If the plasmid is delivered to a non-target cells that lack the miRNA, the repressor/inhibitor is produced and TNFα is not expressed.

This targeting system could be used not only for eliminating harmful cells such as cancers, but used for targeting specific cells or tissues for expressing beneficial genes. An example would be a plasmid encoding vascular endothelial growth factor (VEGF). When attempting to express this gene it may be desirable to only target a limited region so as not to over produce a large number of blood vessels. The same process could be used to limit the target cells by including a specific miRNA-binding site in the plasmid to prevent the expression of VEGF in non-target cells.

These plasmids could also be used in combination with existing antisense technology to produce a system in which expression can be regulated by delivering molecules to the cells that interfere with miRNA function or expression, such as antisense molecules. While these antisense molecules are intact they prevent a prevent production of a specific miRNA or inhibit binding of the miRNA to the miRNA-binding site in the gene of interest, which in turn allows for the expression of the gene of interest. After the antisense molecules degrade or are removed, the miRNAs can then bind to the binding site on the plasmid and inhibit expression of the gene of interest. For instance, in the case of a plasmid expressing VEGF, it would be undesirable to-have the plasmid expressing for an extended period as this may result in production of a hemangioma. By limiting duration of its expression, this can be overcome.

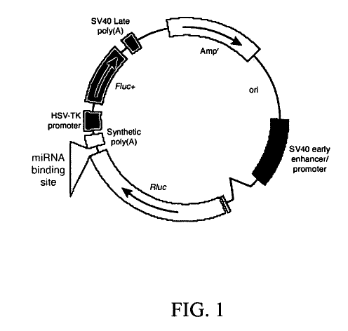

The combination of the expression plasmid with delivery of an antisense molecule could also be used to form an inducible expression plasmid. Take, for example, erythropoietin (EPO), a protein that causes an increase in red blood cell production and is used to treat anemia. Over production of EPO causes a thickening of the blood and has deleterious effects. If a plasmid that expresses EPO and has the miR-122 binding site as in FIG. 1, were delivered to the liver of an anemic individual, it would not express. When a miR-122 antisense molecule that inhibits miR-122 expression of function is delivered to those cells, EPO would be expressed only until the antisense molecules are degraded or removed. If in the future the individual becomes anemic again, the miR-122 antisense molecule could again be delivered and inhibition of EPO expression would be relieved.

The term polynucleotide, or nucleic acid, is a term of art that refers to a polymer containing at least two nucleotides. Nucleotides are, the monomeric units of polynucleotide polymers. Polynucleotides with less than 120 monomeric units are often called oligonucleotides. Natural nucleic acids have a deoxyribose- or ribose-phosphate-backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, β-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations on DNA, RNA and other natural and synthetic nucleotides.

A delivered polynucleotide can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

The polynucleotide may contain sequences that do not serve a specific function in the target cell but are used in the generation of the polynucleotide. Such sequences include, but are not limited to, sequences required for replication or selection of the polynucleotide in a host organism.

A transfection reagent or delivery vehicle is a compound or compounds that bind(s) to or complex(es) with an inhibitor and mediates its entry into cells. Examples of transfection reagents include, but are not limited to, non-viral vectors, cationic liposomes and lipids, polyamines, calcium phosphate precipitates, histone proteins, polyethylenimine, and polylysine complexes. A non-viral vector is defined as a vector that is not assembled within an eukaryotic cell including protein and polymer complexes (polyplexes), lipids and liposomes (lipoplexes), combinations of polymers and lipids (lipopolyplexes), and multilayered and recharged particles. It has been shown that cationic proteins like histones and protamines, or synthetic polymers like polylysine, polyarginine, polyornithine, DEAE dextran, polybrene, and polyethylenimine may be effective intracellular delivery agents. Typically, the transfection reagent has a component with a net positive charge that binds to the oligonucleotide's or polynucleotide's negative charge. The transfection reagent mediates binding of oligonucleotides and polynucleotides to cells via its positive charge (that binds to the cell membrane's negative charge) or via ligands that bind to receptors in the cell. For example, cationic liposomes or polylysine complexes have net positive charges that enable them to bind to DNA or RNA.

A polynucleotide-based gene expression inhibitor comprises any polynucleotide containing a sequence whose presence or expression in a cell causes the degradation of or inhibits the function, transcription, or translation of a gene in a sequence-specific manner. Polynucleotide-based expression inhibitors may be selected from the group comprising: siRNA, microRNA, interfering RNA or RNAi, dsRNA, ribozymes, antisense polynucleotides, and DNA expression cassettes encoding siRNA, microRNA, dsRNA, ribozymes or antisense nucleic acids. RNAi molecules are polynucleotides or polynucleotide analogs that, when delivered to a cell, inhibit RNA function through RNA interference. Small RNAi molecules include RNA molecules less that about 50 nucleotides in length and include siRNA and miRNA. SiRNA comprises a double stranded structure typically containing 15-50 base pairs and preferably 19-25 base pairs and having a nucleotide sequence identical or nearly identical to an expressed target gene or RNA within the cell. An siRNA may be composed of two annealed polynucleotides or a single polynucleotide that forms a hairpin structure. MicroRNAs (miRNAs) are small noncoding polynucleotides that direct destruction or translational repression of their mRNA targets. Antisense polynucleotides comprise sequence that is complimentary to a gene or mRNA. Antisense polynucleotides include, but are not limited to: morpholinos, 2′-O-methyl polynucleotides, DNA, RNA and the like. The polynucleotide-based expression inhibitor may be polymerized in vitro, recombinant, contain chimeric sequences, or derivatives of these groups. The polynucleotide-based expression inhibitor may contain ribonucleotides, deoxyribonucleotides, synthetic nucleotides, or any suitable combination such that the target RNA and/or gene is inhibited.

### EXAMPLES

### Example 1

### Plasmid Constructs

A. Short term miRNA sensor plasmids. Short term miRNA sensor plasmids were created by cloning miRNA binding sites into the 3′ UTR of the Renilla luciferase gene in the PSICHECK™-2 Vector (Promega, Madison, Wis., GenBank accession #AY535007, FIG. 1). This vector contains the Renilla luciferase and firefly luciferase genes under the control of separate enhancer/promoters. The firefly luciferase (Photinus pyralis) gene serves as an internal control which permits normalization of delivery efficiency.

A subset of 40 known miRNAs was chosen and their exact complements and respective antisense sequences were synthesized (IDT, Coralville, Iowa). Sequences of mature mouse miRNAs were acquired from the miRBase Sequence Database (Wellcome Trust Sanger Institute, United Kingdom). Equal molar amounts of each oligonucleotide pair were annealed and ligated into the PSICHECK™-2 plasmid. A subset of these, for microRNAs miR-122, miR-18, miR-192, miR-143 and miR-1,. are listed in Table 1. The miRNA binding site sequences were cloned into the XhoI/NotI sites located in the 3′ untranslated region of Renilla luciferase gene.

TABLE 1

| | |
|---|---|
| oligonucleotide | sequence |

| | |
|---|---|
| MRT-122S | TCGAGACAAACACCATTGTCACACTCCAGC |
| MRT-122AS | GGCCGCTGGAGTGTGACAATGGTGTTTGTC |
| MRT-192S | TCGAGGGCTGTCAATTCATAGGTCAGGC |
| MRT-192AS | GGCCGCCTGACCTATGAATTGACAGCCC |
| MRT-1S | TCGAGTACATACTTCTTTACATTCCAGC |
| MRT-1AS | GGCCGCTGGAATGTAAAGAAGTATGTAC |
| MRT-18S | TCGAGTATCTGCACTAGATGCACCTTAGC |
| MRT-18AS | GGCCGCTAAGGTGCATCTAGTGCAGATAC |
| MRT-143S | TCGAGTGAGCTACAGTGCTTCATCTCAGC |
| MRT-143AS | GGCCGCTGAGATGAAGCACTGTAGCTCAC |

MRT = miRNA binding site oligonucleotide; numbers refer to the miRNA as listed in the Sanger Institute miRNA Registry.
S = sense strand containing sequence complementary to that of corresponding endogenous miRNA according to standard convention.
AS = antisense strand which contains sequence complementary to the corresponding sense strand.

TABLE 2
Short term miRNA sensor plasmids

| Plasmid | binding site oligo | miRNA |
|---|---|---|
| pMIR393 | none | — |
| pMIR394 | MRT-18 | miR-18 |
| pMIR395 | MRT-192 | miR-192 |
| pMIR398 | MRT-143 | miR-143 |
| pMIR399 | MRT-122 | miR-122a |
| pMIR400 | MRT-1 | miR-1 |

B. Long Term miRNA Sensor Plasmids.

Plasmids were created to enable long-term monitoring of miRNA expression in mouse liver. In order to achieve long term monitoring of miRNA expression in mouse liver, enhancer/promoter combinations that give long-term expression of a reporter gene in the liver are required. It is well known in the field that viral enhancer/promoter combinations, such as the SV-40 early enhancer/promoter present in the PSICHECK™-2 vector used in the short term miRNA sensor plasmid are largely inactivated in the liver within about 24 hours after delivery. Although viral enhancer/promoter combinations such as this drive short-term high-levels of reporter gene expression in liver, they are not useful in longer-term studies.

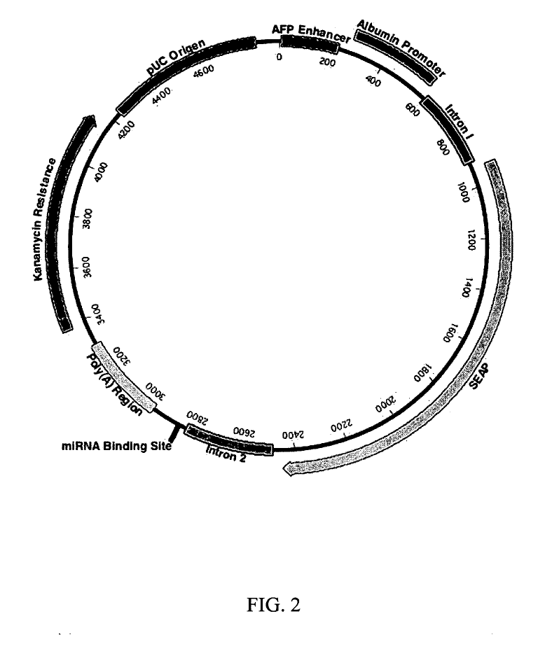

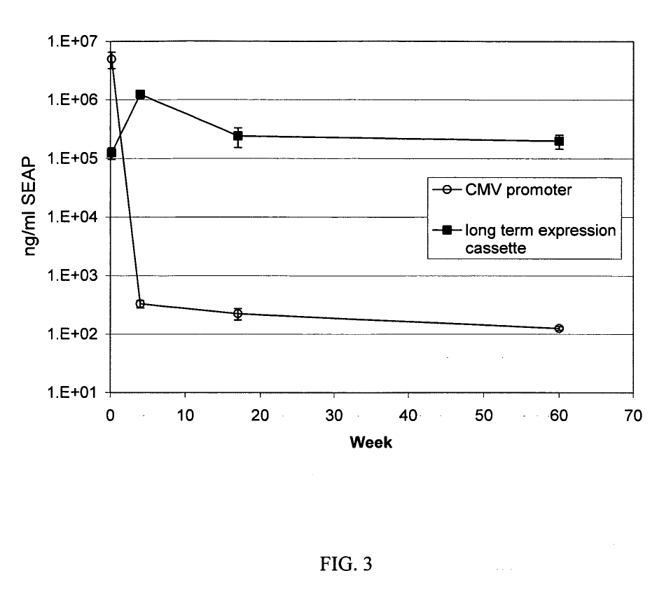

We have developed novel plasmids containing transcriptional control elements that allow long-term expression in mouse liver. These long term expression plasmids utilize a chimeric promoter composed of the minimal mouse albumin promoter and the mouse alpha-fetoprotein enhancer II (U.S. patent application Ser. No. 10/229,786). These transcriptional control elements are liver specific, thus enabling liver-specific expression after delivery by HTV injection. Two introns, a 5′ intron and a 3′intron, have been engineered into the expression plasmids such that they are present in the primary transcript (U.S. patent application Ser. No. 10/229,786). These long term expression plasmids (FIG. 2) give high, sustained levels of human SEAP (hSEAP) expression for at least 14 months (FIG. 3) in C57B1/6 mouse hepatocytes after HTV injection. In contrast, hSEAP expression driven by the CMV immediate early viral enhancer/promoter was reduced to very low levels by Day 7 post-injection.

The hSEAP gene, which is highly similar to the murine SEAP, is not immunogenic in the C57B1/6 inbred mouse strain. However, hSEAP is immunogenic in more outbred mouse strains such as ICR. In order to achieve ling term expression in more outbred strains of mice, the native mouse SEAP is used instead of human SEAP. We have confirmed that murine SEAP (mSEAP) is non-immunogenic in ICR mice by delivering a plasmid that contains the mSEAP gene under the transcriptional control of the CMV enhancer/promoter (CMV-mSEAP). This vector was delivered to muscle by HLV injection. Unlike in liver, the CMV enhancer/promoter is not shut down in muscle tissue. Expression data from this plasmid shows long-term expression of mSEAP in mouse skeletal muscle. In contrast, expression of human SEAP from the CMV enhancer/promoter is suppressed after 14 days in muscle, concomitant with the appearance of anti-SEAP antibodies. Together these data indicate that mSEAP is non-immunogenic in mice. Because SEAP is secreted into the bloodstream, it enables the investigator to monitor its expression by taking blood samples, such as from retro-orbital bleed, and using simple assays for quantitative detection. mSEAP expression can be assayed using commercially available chemiluminescent reagents readily available in the art.

A soluble version of CD4 (smCD4) can be used as an alternative reporter gene or as an internal delivery control. As an internal control, smCD4 can be located on the same plasmid as the reporter gene or on a separate plasmid. SmCD4 can be assayed by ELISA using commercially available antibodies (Abcam).

Anti-miRNA antisense oligonucleotides. For inhibition of miRNA function, antisense oligonucleotides containing 2′OCH₃ substituted, morpholino or locked nucleotides were synthesized. The control antisense oligonucleotide, GL-3ome, is not complementary to any of the miRNAs. Sequences of the anti-miRNA antisense oligonucleotides (5′-3′) are shown in Table 3.

TABLE 3

| | |
|---|---|
| Anti-miRNA oligo | sequence |

| | |
|---|---|
| MRT-122ome | ACAAACACCAUUGUCACACUCCA |
| MRT-122morph | ACAAACACCATTGTCACACTCCA |
| MRT-1221na | ACA+AA+CA+CC+AT+TG+TC+AC+AC+TC+CA |
| MRT-192ome | GGCUGUCAAUUCAUAGGUCAG |
| MRT-1ome | UACAUACUUCUUUACAUUCCA |
| MRT-18ome | UAUCUGCACUAGAUGCACCUUA |
| MRT-143ome | UGAGCUACAGUGCUUCAUCUCA |
| GL-3ome | CUUACGCUGAGUACUUCGAUU |

MRT = miRNA target; numbers refer to the miRNA as listed in the Sanger Institute miRNA Registry.
‘ome’ indicates oligonucleotide contains 2′OCH₃ (2′OMe) substitutions.
‘morph’ indicates oligonucleotide is composed entirely of morpholino nucleotides.
‘1na’ indicates oligonucleotide contains locked nucleic acids.
‘+’ indicated position of locked nucleotides (methylene linkage between the 2′ and 4′ positions of the ribose).
GL-3ome, control antisense oligonucleotides.

### Example 2

### Plasmid Delivery and Reporter Protein Assays

A. Mouse hydrodynamic tail vein injections and dual luciferase assay. Approximately 20 g ICR mice (Harlan-Sprague Dawley) were injected in the tail vein with 10 μg of plasmid DNA with or without 10 μg of miRNA inhibitory antisense oligonucleotide in 2 ml Ringer's solution (1 ml per 10 grams body weight) in 5-7seconds according to the hydrodynamic delivery method (U.S. Pat. No. 6,627,616) for delivery to hepatocytes. The liver was harvested and homogenized one day after injection. The homogenate was assayed for Renilla luciferase and firefly luciferase activity using the Dual Luciferase Assay (Promega Corp. Madison, Wis.) and the ratio of Renilla luciferase to firefly luciferase calculated. Data was normalized to animals receiving the PSICHECK™-2 vector without miRNA binding sites, parent vector pMIR393.

B. Mouse hydrodynamic limb vein injections and dual luciferase assay. ICR mice were injected in the saphenous vein with 20 μg of plasmid DNA or 20 μg plasmid DNA+20 μg siRNA in 1 ml 0.9% saline solution at a rate 8 mls/minute according to the intravenous delivery method (U.S. patent application Ser. No. 10/855,175, incorporated herein by reference) for delivery of the miRNA sensor plasmid to limb skeletal muscle. The skeletal muscle was harvested and homogenized two days after injection. The homogenate (without dilution or diluted 1:10) was assayed for Renilla luciferase and firefly luciferase activity using the Dual Luciferase Assay (Promega Corp. Madison, Wis.) and the ratio amount of Renilla luciferase to firefly luciferase calculated. Data was normalized to animals receiving the PSICHECK™-2 vector without miRNA binding sites, parent vector pMIR393.

C. HeLa cell transfection and dual luciferase assay. HeLa cells were grown to 50% confluency in 24 well plates in DMEM/10% FBS with Pen/Strep and transfected in triplicate with plasmid DNA (0.25 μg/well) using TRANSIT-LT1® at a 3:1 ratio (Mirus Bio Corporation, Madison, Wis.). Two hours later, miRNA inhibitory antisense oligonucleotide or a control oligonucleotide was transfected using TRANSIT-OLIGO® (Mirus Bio Corporation, Madison, Wis.; 1 μl/well, 100 nM oligonucleotide/well). The cells were harvested 24 hours later and the amount of Renilla luciferase and firefly luciferase activity was measured using the Dual Luciferase Assay. Data was normalized to animals receiving the PSICHECK™-2 vector without miRNA binding sites, parent vector pMIR393.

### Example 3

### Short Term miRNA Sensor Plasmids Effectively Monitor miRNA Activity in Mouse Liver

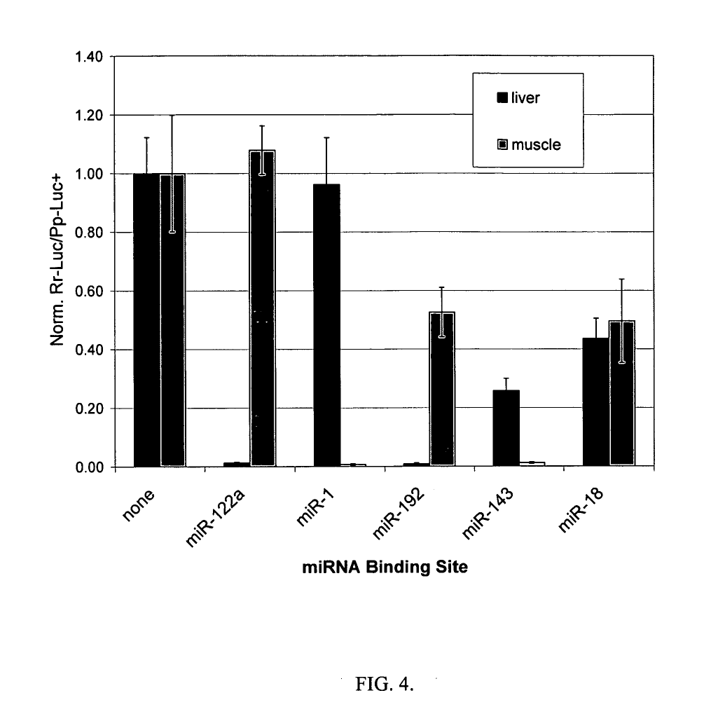

Short term miRNA sensor plasmids pMIR393, pMIR394, pMIR395, pMIR398, pMIR399, and pMIR400 (see Table 2) were delivered to hepatocytes in vivo as described above. Renilla luciferase expression in the liver from the miRNA sensor plasmids is shown in FIG. 4. According to published data, miR-122a and miR-192 are highly expressed in liver and but have not been detected in skeletal muscle. The presence of the miR-122a and miRNA192 binding sites resulted in nearly complete inhibition of expression of the reporter gene. The presence of the miR-1 binding site did not result in inhibition of reporter gene expression. The presence of the miR-143 and miR-1 8 binding sites resulted in 74.2% and 56% inhibition of reporter gene expression, respectively.

### Example 4

### Short Term miRNA Sensor Plasmids Effectively Monitor miRNA Activity in Mouse Skeletal Muscle

Short term miRNA sensor plasmids pMIR393, pMIR394, pMIR395, pMIR398, pMIR399, and pMIR400 (see Table 2) were delivered to limb skeletal muscle cells as described above. Renilla luciferase expression in the liver from the miRNA sensor plasmids is shown in FIG. 4. According to published data, miR-1 has been found to be highly expressed in skeletal muscle, but has not been detected in liver. The presence of the miR-1 binding sites in the 3′UTR of the reporter gene resulted in nearly complete inhibition of expression of the reporter gene, less than 1% of expression level compared to reporter gene without the miR-1 binding site. This results indicates that miR-1 is expressed and functional in mouse skeletal muscle. The presence of the miR-122a binding site did not result in inhibition of reporter gene expression in skeletal muscle. This result indicates that miR-122 is not functional in mouse skeletal muscle.

### Example 5

### miRNA Function Detection is Dependent on an Appropriate miRNA Binding Site

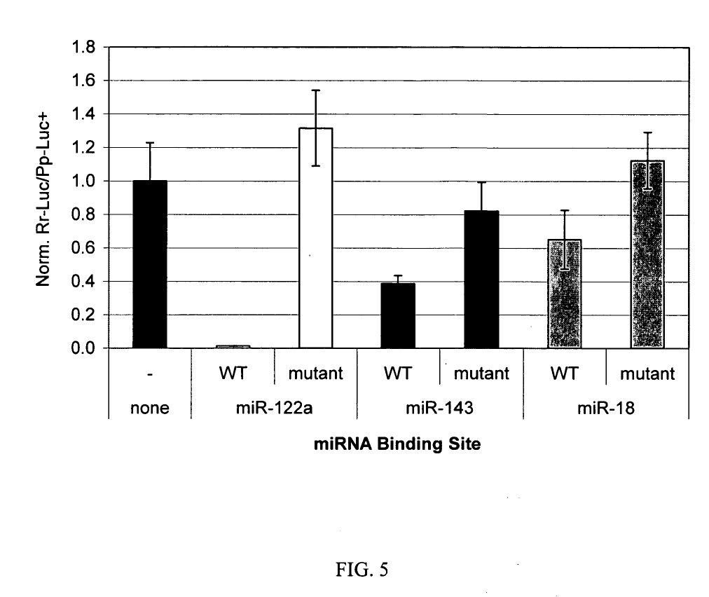

In order to test the specificity of miRNA sensor plasmids in mice, we constructed sensor plasmids for miR-122a, miR-143 and miR-18 with mutations at miRNA binding site positions corresponding to positions 3, 7 and 10 of the cognate miRNA. Positions 3 and 7 are in the seed-region of the miRNA and position 10 has been shown to be important for mRNA cleavage. We delivered 10 μg of sensor plasmid to each mouse by HTV injection. Livers were harvested 24 hours post injection and extracts were examined for luciferase activity. As shown in FIG. 5, the mutations in the miRNA binding sites relieved the inhibition by all three miRNAs, providing evidence that the inhibition is miRNA binding site-specific.

### Example 6

### Analysis of Effectiveness of miRNA Sensor Plasmids on Determined Endogenous miRNA Function

According to published data, miR-122a and miR-192 are highly expressed in liver and expression in skeletal muscle is not detected, while miR-1 is highly expressed in skeletal muscle and not detected in liver. As shown in FIG. 4, our reporter assay data correlated well with the published data with nearly complete inhibition of Renilla expression in the appropriate tissue. The sensor plasmids designed to detect miR-122a and miR-192 showed inhibition of reporter gene expression only in liver while retaining maximal expression in muscle. Similarly, the sensor plasmid designed to detect the presence and function of miR-1 showed nearly complete inhibition of reporter gene expression in skeletal muscle, with no inhibition in liver. Microarray and Northern data have indicated that expression of miR-143 is higher in liver than in muscle. The results from the sensor plasmid again correlates directly with this experimental evidence. The miR-18 sensor plasmid showed a moderate level of miR-1 8 activity in both liver and muscle. However, this miRNA has not been previously detected in these tissues by the microarray or Northern analyses. The miRNA sensor plasmid therefore appears to be more sensitive than these microarray and Northern analyses in detected miRNAs.

### Example 7

### Short Term miRNA Sensor Plasmids Effectively Monitor miRNA Activity in Cells in Culture

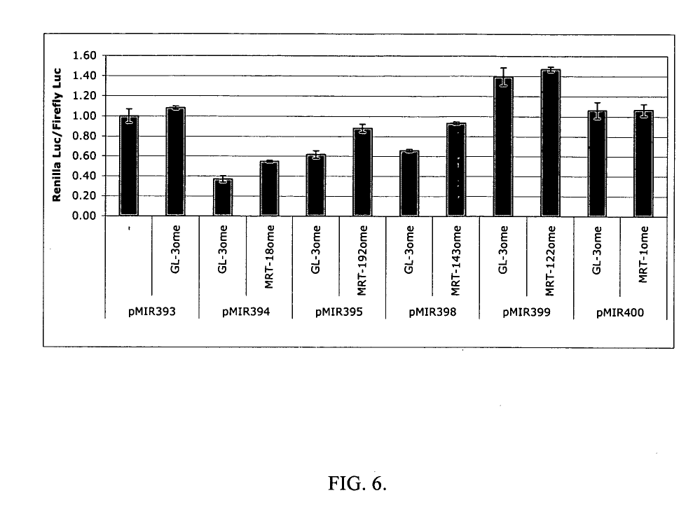

Cells were transfected with plasmids containing sequences complementary to miR-18 (pMIR394), miR-192 (pMIR395), miR-1⁴3 (pMIR398), miR-122 (pMIR399), or miR-1 (pMIR400), or with the parent plasmid (pMIR393). Two hours later, the cells were transfected with anti-miRNA antisense oligonucleotides (MRT), control oligonucle otide (GL-3 ome), or no oligonucleotide (−). Cells were harvested one day later and assayed for Renilla and firefly Luc. Results, shown in FIG. 6, indicate that Renilla Luc expression was inhibited in cells transfected pMIR394, pMIR395 and pMIR398 relative to cells transfected with pMIR393. These results indicate that miR-18, miR-192 and miR-143 are expressed and functional in HeLa cells. Addition of the corresponding anti-miRNA antisense oligonucleotide relieved the inhibition. No inhibition of Renilla Luc expression was observed in cells transfected with pMIR399 or pMIR400 indicating that miR-122 and miR-1 are not expressed or are not functional in HeLa cells.

### Example 8

### 2′-OMe Substituted Antisense Oligonucleotides can Inhibit miRNA Function in Liver

Studies have shown that miRNAs can be inhibited by oligonucleotides containing 2′-O-methyl (2′-OMe) substitutions having the antisense sequence to the mature miRNA. Inhibition was shown to be due to binding to the miRNA. In order to further test the specificity of the miRNA sensor plasmids, 10 mg of the miRNA sensor plasmids containing the binding sites for miR-18, 143, and 122 were co-delivered to liver by HTV injection with 10 mg of either the indicated 2′-OMe antisense oligonucleotide or a non-specific antisense control. The control plasmid, pMIR393, was delivered in control mice. Liver tissue were harvested one day after injection and extracts assayed for luciferase activity.

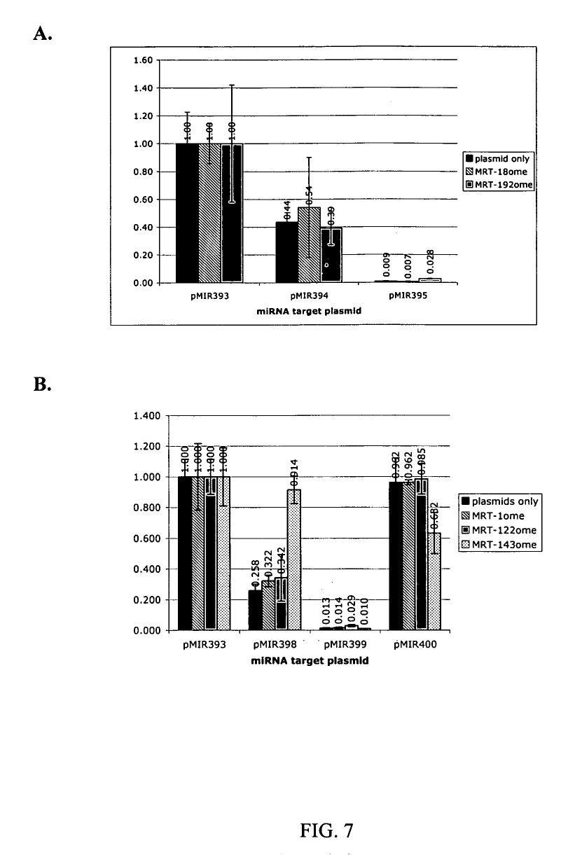

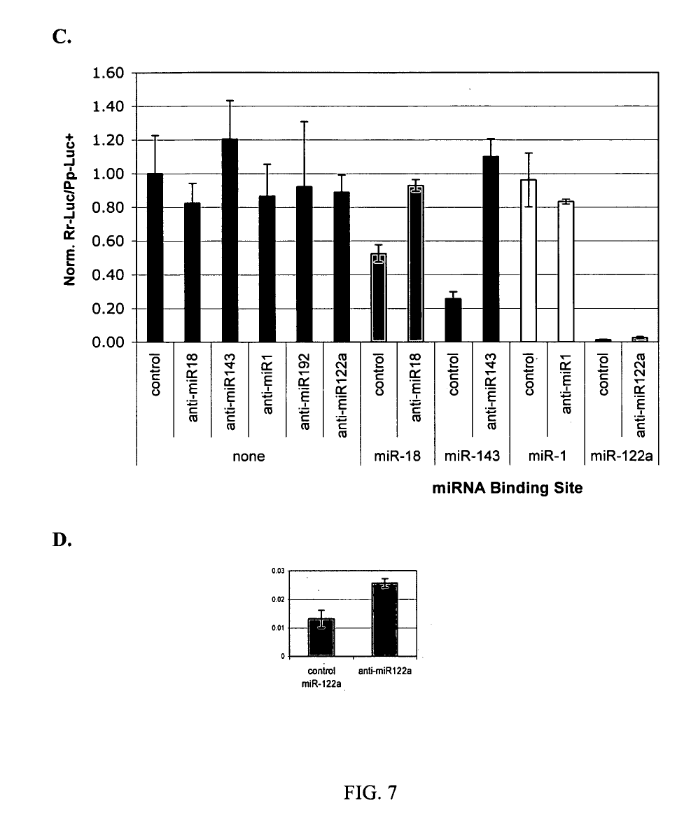

2′-OMe antisense oligonucleotides to the miRNAs were able to provide total relief of inhibition when co-delivered with miR-18 and miR-143 sensor plasmids. In the case of miR-122a, inhibition was evident but incomplete (FIG. 7). Incomplete inhibition by 2′-OMe antisense miRNA could be due to high levels of this miRNA in liver. It has been reported that miR-122a is highly expressed in hepatocytes, with more than 50,000 copies per cell. The copy numbers of the other miRNAs shown in FIG. 7 have not been reported.

Co-delivery the 2′-OMe antisense oligonucleotides MRT-18ome, MRT-143ome, MRT-192ome, and MRT-122ome, resulted in increased expression of relative Renilla Luc from their cognate miRNA sensor genes, but not from reporter genes containing a different miRNA binding sites. Co-delivery of MRT-1 ome did not have an effect on relative Renilla Luc expression from pMIR393, pMIR398, pMIR399 or pMIR400. These results demonstrate the specificity miRNA in inhibiting expression from the cognate miRNA sensor gene.

### Example 9

### Comparison of Antisense Chemistries for miRNA Inhibition in vivo

The in vivo effectiveness of antisense miRNA inhibitors with 2′-OMe, morpholino and locked nucleic acid (LNA) antisense modifications was tested against the highly expressed liver miRNA, miR-122a. pMIR399 was delivered to mouse hepatocytes along with different anti-miR122a oligonucleotides as described above.

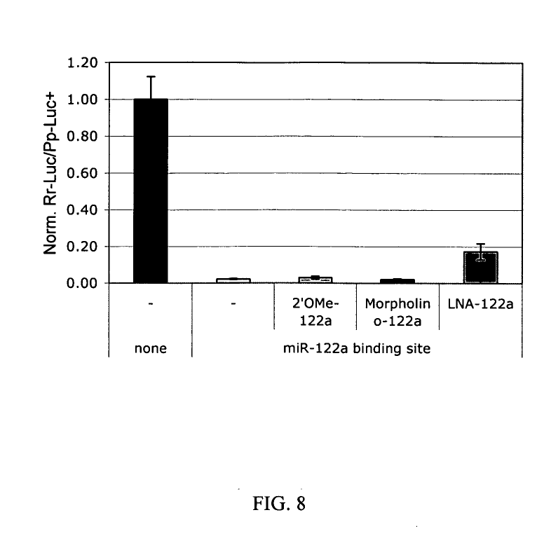

Morpholino oligomers are uncharged nucleotide analogs in which a six-membered morpholine ring is substituted for the sugar moiety and a non-ionic phosphorodiamidate linkage replaces the typical phosphodiester linkage. LNAs contain a bridge between the 2′-O and the 4′-position via a methylene linker that “locks” it into a C3′-endo (RNA) sugar conformation. LNAs have been used previously to inhibit miRNA function. The results are shown in FIG. 8. The morpholino oligonucleotide had no effect in relieving inhibition of sensor plasmid target gene expression. In contrast, the LNA modification was 1 0-fold more effective than the 2′-OMe substituted oligonucleotide, resulting in recovery of miRNA sensor reporter gene activity to 20% of control levels. Together with the mutant binding site data shown in FIG. 5, the greater ability of the LNA antisense oligonucleotide for relieving inhibition is evidence that the lack of complete relief of inhibition of target gene expression is not due to factors other than miRNA-induced cleavage, but rather is likely due to the high copy number of miR-122a in hepatocytes. These data indicate that the described miRNA sensor plasmids can be used to test the effectiveness of different antisense oligonucleotides or oligonucleotides chemistries in inhibiting miRNA activity.

### Example 10

### miRNAs are Differentially Expressed between Fed and Fasted Mice

Modulation of the expression of particular miRNAs in response to different treatments would provide clues as to their function. Using mirMAX X-Species miRNA microarrays (Bionomics Research & Technology Center, Rutgers University) and competitive hybridization to compare miRNA expression in mouse liver from fed and fasted animals, it was observed that 6 of 36 liver expressed miRNAs were differently expressed in fasted vs. fed livers. 5 of the 6 differed by more than ˜10-fold, with one displaying ˜100-fold difference between fed and fasted mice. For these analyses, three ICR mice were fasted for 36 hours and three ICR mice were fed ad libidum. All mice had free access to water. At the end of the fasting period, livers were harvested and small RNAs were isolated from 0.25 mg of tissue using the mirVana miRNA Isolation Kit (Ambion, Austin, Tex.). Using the described long term miRNA sensor plasmids, the onset of differential expression can be monitored.

### Example 11

### miRNA Analysis Library

The described long-term miRNA sensor plasmids can be used to create a miRNA sensor library. A sensor library contains a set of miRNA sensor plasmids with miRNA binding sites for each of the known miRNAs. Subsets of the library can contain sensor plasmids to detect each of the known miRNAs in a specific tissue for a specific animal species, such as mouse liver. It is further possible to create miRNA sensor plasmids with suspected miRNA binding sites. It is also possible to create miRNA sensor plasmids that contain multiple miRNA binding sites which may be the same or different. The multiple binding sites may contain exact match miRNA binding sites or sites which are not exact matches. For instance, some endogenous genes contain multiple non-exact match miRNA binding sites. It is possible to insert the miRNA regulatory region of the endogenous gene into the long term expression miRNA sensor plasmid to provide a means to readily investigate the role of miRNAs in regulating the endogenous gene. Sequences for known miRNAs can be found in databases such as the miRBase Sequence Database from the Wellcome Trust Sanger Institute.

### Example 12

### mSEAP Long-Term miRNA Sensor Plasmid

In one embodiment, an long term liver miRNA sensor plasmid comprises the mSEAP reporter gene driven by the minimal mouse albumin promoter and the mouse alpha-fetoprotein enhancer II and containing a 5′ intron, 3′ intron and a 3′ UTR exact match miRNA binding site. The miRNA sensor plasmid is delivered to hepatocytes in a mouse by HTV injection. As a control for delivery efficiency, a second long term expression plasmid encoding the CD4 gene is co-delivered with the miRNA sensor plasmid. Verification of miRNA activity results obtained through monitoring expression of the reporter gene are confirmed by delivering antisense miRNA oligonucleotides or by delivering an mSEAP expression plasmid without the miRNA binding site.

We claim:

1. A miRNA sensor gene for detecting activity of an endogenous miRNA in vivo comprising: a long term promoter/enhancer, a secreted reporter gene and a miRNA binding site.

2. The miRNA sensor gene of claim 1 further comprising a 5′ intron.

3. The miRNA sensor gene of claim 2 further comprising a 3′ intron.

4. The miRNA sensor gene of claim 3 wherein the miRNA binding site is located in the 3′ untranslated region (UTR) of the reporter gene.

5. The miRNA sensor gene of claim 4 wherein the sensor gene comprises a secreted alkaline phosphatase gene.

6. The miRNA sensor gene of claim 5 wherein the secreted alkaline phosphatase comprises murine secreted alkaline phosphatase.

7. The miRNA sensor gene of claim 4 wherein the miRNA binding site consists of a perfect match miRNA binding site.

8. The miRNA sensor gene of claim 4 wherein the miRNA binding site consists of a partially complementary miRNA binding site.

9. The miRNA sensor gene of claim 4 wherein the partially complementary miRNA binding site contains perfect complementarity to a seed region of the miRNA.

10. The miRNA sensor gene of claim 1 wherein the sensor gene contains a plurality of miRNA binding sites.

11. A process for analyzing activity of an endogenous miRNA in a hepatocyte in a mouse in vivo comprising: a) forming a miRNA sensor plasmid comprising: an a-fetoprotein enhancer, an albumin promoter, a 5′ intron, a murine secreted alkaline phosphatase reporter gene, a 3′ intron and a 3′ miRNA binding site;
b) delivering the sensor plasmid to the hepatocyte by hydrodynamic tail vein injection;
and,
c) monitoring the level of secreted alkaline phosphatase in the blood of the mouse.

12. The process of claim 11 wherein the 3′ miRNA binding site consists of a perfect match miRNA binding site.

13. The process of claim 11 wherein the 3′ miRNA binding site consists of a partially complementary miRNA binding site.

14. The process of claim 13 wherein the partially complementary miRNA binding site contains perfect complementarity to a seed region of the miRNA.

15. The process of claim 1 wherein the miRNA sensor plasmid contains a plurality of miRNA binding sites.

16. A miRNA sensor library consisting of a set of miRNA sensor plasmids wherein each miRNA sensor plasmid comprises: an a-fetoprotein enhancer, an albumin promoter, a 5′ intron, a murine secreted alkaline phosphatase reporter gene, a 3′ intron and a unique 3′ miRNA binding site.

17. The miRNA sensor library of claim 16 wherein the 3′ miRNA binding consists of a perfect match miRNA binding site.

18. The miRNA sensor library of claim 17 wherein the each miRNA sensor plasmid further comprises a control reporter gene.

19. The miRNA sensor library of claim 17 wherein the set of miRNA sensor plasmids comprises miRNA sensor plasmids for endogenous miRNAs known to be present in a desired tissue or cell type or at a developmental stage.

20. A mouse transfected with a liver-specific long term expression miRNA sensor plasmid.


SEQUENCE LISTING (17 sequences)

SEQ ID NO: 1 - DNA, 30 bp, Mus musculus
tcgagacaaacaccattgtcacactccagc

SEQ ID NO: 2 - DNA, 30 bp, Mus musculus
ggccgctggagtgtgacaatggtgtttgtc

SEQ ID NO: 3 - DNA, 28 bp, Mus musculus
tcgagggctgtcaattcataggtcaggc

SEQ ID NO: 4 - DNA, 28 bp, Mus musculus
ggccgcctgacctatgaattgacagccc

SEQ ID NO: 5 - DNA, 28 bp, Mus musculus
tcgagtacatacttctttacattccagc

SEQ ID NO: 6 - DNA, 28 bp, Mus musculus
ggccgctggaatgtaaagaagtatgtac

SEQ ID NO: 7 - DNA, 29 bp, Mus musculus
tcgagtatctgcactagatgcaccttagc

SEQ ID NO: 8 - DNA, 29 bp, Mus musculus
ggccgctaaggtgcatctagtgcagatac

SEQ ID NO: 9 - DNA, 29 bp, Mus musculus
tcgagtgagctacagtgcttcatctcagc

SEQ ID NO: 10 - DNA, 29 bp, Mus musculus
ggccgctgagatgaagcactgtagctcac

SEQ ID NO: 11 - RNA, 23 nt, Mus musculus
acaaacaccauugucacacucca

SEQ ID NO: 12 - DNA, 23 bp, Mus musculus
acaaacaccattgtcacactcca

SEQ ID NO: 13 - RNA, 21 nt, Mus musculus
ggcugucaauucauaggucag

SEQ ID NO: 14 - RNA, 21 nt, Mus musculus
uacauacuucuuuacauucca

SEQ ID NO: 15 - RNA, 22 nt, Mus musculus
uaucugcacuagaugcaccuua

SEQ ID NO: 16 - RNA, 22 nt, Mus musculus
ugagcuacagugcuucaucuca

SEQ ID NO: 17 - RNA, 21 nt, Photinus pyralis
cuuacgcugaguacuucgauu